SEQUENCE LISTING (32 sequences)

SEQ ID NO: 1 - DNA, 22 bp, Artificial
  forward primer for PCR
aatcagacggctagacgagagg

SEQ ID NO: 2 - DNA, 22 bp, Artificial
  reverse primer for PCR
tcagcagatttaaggccctcat

SEQ ID NO: 3 - DNA, 24 bp, Artificial
  forward primer for PCR
acgcgtgtgagaaagaatacagac

SEQ ID NO: 4 - DNA, 20 bp, Artificial
  reverse primer for PCR
gcttgagtttggttctgggc

SEQ ID NO: 5 - DNA, 22 bp, Artificial
  forward primer for PCR
tggaaaattctgcgagtgtgat

SEQ ID NO: 6 - DNA, 22 bp, Artificial
  reverse primer for PCR
tgccagtgtaattgggatagca

SEQ ID NO: 7 - DNA, 21 bp, Artificial
  forward primer for PCR
gcgaaggataatagcaggcat

SEQ ID NO: 8 - DNA, 20 bp, Artificial
  reverse primer for PCR
ttgtcacccgaggtcccata

SEQ ID NO: 9 - DNA, 26 bp, Artificial
  forward primer for PCR
caaaaatgtatcacaccaaaggacaa

SEQ ID NO: 10 - DNA, 25 bp, Artificial
  reverse primer for PCR
tgcttggtacacacttgaccttctt

SEQ ID NO: 11 - DNA, 20 bp, Artificial
  forward primer for PCR
gtggatgccgatgaactgac

SEQ ID NO: 12 - DNA, 20 bp, Artificial
  reverse primer for PCR
acagcgttccaatcaccaaa

SEQ ID NO: 13 - DNA, 22 bp, Artificial
  forward primer for PCR
tatcgcctacaaccagaccatc

SEQ ID NO: 14 - DNA, 23 bp, Artificial
  reverse primer for PCR
gggtgcgtacatagagcataaga

SEQ ID NO: 15 - DNA, 19 bp, Artificial
  forward primer for PCR
cccactcacgggtgtcaaa

SEQ ID NO: 16 - DNA, 20 bp, Artificial
  reverse primer for PCR
tgctccactgagctcccatt

SEQ ID NO: 17 - DNA, 17 bp, Artificial
  forward primer for PCR
tggcttggcggtgtgat

SEQ ID NO: 18 - DNA, 21 bp, Artificial
  reverse primer for PCR
cacacgggacaattgagttca

SEQ ID NO: 19 - DNA, 22 bp, Artificial
  forward primer for PCR
cagcacgaaggatgctgataac

SEQ ID NO: 20 - DNA, 22 bp, Artificial
  reverse primer for PCR
ttgtcccgcagtgtagaacatt

SEQ ID NO: 21 - DNA, 20 bp, Artificial
  forward primer for PCR
aagcatgcccatctggttac

SEQ ID NO: 22 - DNA, 20 bp, Artificial
  reverse primer for PCR
gcctgccttctttctcacac

SEQ ID NO: 23 - DNA, 20 bp, Artificial
  forward primer for PCR
gctctgcatcagtgacggta

SEQ ID NO: 24 - DNA, 20 bp, Artificial
  reverse primer for PCR
taattacgggtcaatgcaca

SEQ ID NO: 25 - DNA, 21 bp, Artificial
  forward primer for PCR
ctttgtcatcacactcccctt

SEQ ID NO: 26 - DNA, 21 bp, Artificial
  reverse primer for PCR
gcccacatacactgccttttc

SEQ ID NO: 27 - DNA, 19 bp, Artificial
  forward primer for PCR
ccagacggaacagagcaag

SEQ ID NO: 28 - DNA, 18 bp, Artificial
  reverse primer for PCR
ctgtcctcgtgctgccat

SEQ ID NO: 29 - DNA, 20 bp, Artificial
  forward primer for PCR
cctgcactggagggaggtct

SEQ ID NO: 30 - DNA, 20 bp, Artificial
  reverse primer for PCR
ggctccagcaccttccagtc

SEQ ID NO: 31 - DNA, 22 bp, Artificial
  forward primer for PCR
ctacagcaacagggtggtggac

SEQ ID NO: 32 - DNA, 22 bp, Artificial
  reverse primer for PCR
ggatagggcctctcttgctcag

(19) United States
(12) Patent Application Publication
(10) Pub. No.: US 2013/0243736 A1
(43) Pub. Date: Sep. 19, 2013

(54) REPLACEMENT OF BONE MARROW NICHE CELLS FOR TREATMENT OF VARIOUS DISEASES

(71) Applicant: SAPPORO MEDICAL UNIVERSITY, Sapporo-shi (JP)

(72) Inventors: Mineko Fujimiya, Sapporo-shi (JP); Koji Ataka, Sapporo-shi (JP); Hironori Chiba, Sapporo-shi (JP)

(73) Assignee: SAPPORO MEDICAL UNIVERSITY, Sapporo-shi (JP)

(21) Appl. No.: 13/751,352

(22) Filed: Jan. 28, 2013

Related U.S. Application Data
(60) Provisional application No. 61/591,446, filed on Jan. 27, 2012.

(51) Int. Cl. C12N 5/071; A61K 35/32; C12Q 1/68

(52) U.S. Cl. CPC C12N 5/0654; C12Q 1/6883; A61K 35/32
USPC 424/93.7; 435/373; 435/6.12; 435/6.13

(57) ABSTRACT

In vitro 2-dimensional co-culture systems of LT-HSCs and osteoblastic niche cells and methods of establishing them are provided. For example, in certain aspects methods of screening people suffering from a disorder caused by dysfunction of osteoblastic niche cells, using said co-culture systems are described. In further aspects, methods for screening a candidate substance for treatment of a disorder caused by dysfunction of osteoblastic niche cells are provided. The present invention also concerns methods and therapeutic compositions of treating a patient suffering from a disorder caused by dysfunction of osteoblastic niche cells.

### CROSS-REFERENCE TO RELATED APPLICATIONS

The present application claims priority under 35 U.S.C. §119(e) to Provisional Application No. 61/591,446, filed Jan. 27, 2012, the contents of which are incorporated herein by reference in their entirety.

### BACKGROUND OF THE INVENTION

Long-lasting diabetes impairs progenitor cell-dependent tissue repair, which is associated with the dysfunction of hematopoietic stem cells (HSCs) in bone marrow. In human type 2 diabetes, bone marrow-derived circulating CD34⁺ cells are significantly reduced because of reduced numbers of circulating endothelial progenitor cells, which has been proposed as a mechanism behind cardiovascular complications [1]. Diabetic mice were also shown to have diminished numbers of circulating Lin⁻, Sca-1⁺, c-kit⁺ hematopoietic progenitor cells leading to delayed wound closure [2].

The involvement of the stem cell niche or bone marrow microenvironment has been proposed as a means of understanding hematopoietic progenitor cell dysfunction [3]. Specialized microenvironments (niches) support both self-renewal and the differentiation of HSCs, while their maintenance in a quiescent state is essential to protect from stress and to sustain long-term hematopoiesis. HSCs are located in the trabecular endosteum (osteoblastic niche) or sinusoidal perivascular area (vascular niche) [4], the latter including CXCL12-abundant reticular (CAR) cells [5] or nestin⁺ mesenchymal stem cells [6] characterized by high CXCL12 expression [7]. In mouse models of diabetes induced by streptozotocin (STZ) or db/db, CXCL12 mRNA levels in nestin⁺ cells were reduced, and impaired interaction between CXCL12 expressed on nestin⁺ cells and HSC-expressed CXCR4 caused poor mobilization of hematopoietic cells after granulocyte colony-stimulating factor (GCSF) treatment [6, 8].

The osteoblastic niche appears to be more involved than the vascular niche in diabetic stem cell abnormality, because diabetic mice showed significantly reduced numbers of osteoblastic but not nestin⁺ cells [8]. Osteoblasts secrete cytokines/chemokines including GCSF, granulocyte-macrophage colony-stimulating factor (GM-CSF), macrophage colony-stimulating factor (M-CSF), interleukin (IL)-1, IL-6, IL-7 and CXCL12, which support HSC survival and differentiation [9]. They also express molecules that regulate HSC numbers, including angiopoietin, thrombopoietin (TPO), Wnt, Notch and osteopontin (OPN) [10], and adhesion molecules including N-cadherin, vascular cell adhesion molecule1 (VCAM1), intracellular adhesion molecule-1 (ICAM1) and annexin II [9]. Interaction of HSCs with osteoblastic niche cells through cell adhesion molecules and chemokines and their receptors maintains the balance between cell division/proliferation and quiescence [4].

We previously reported that diabetes induces an increase in the infiltration of bone marrow-derived cells in peripheral organs such as the liver, dorsal root ganglia and kidney tubulo-interstitial space, and that abnormal cell fusion between bone marrow-derived cells and parenchymal cells causes chromosomal abnormalities and accelerates apoptosis in diabetes [11-14]. These findings imply that diabetes primarily leads to a dysfunction of the osteoblastic niche which subsequently disrupts HSC quiescence, then increased abnormal bone marrow-derived cells induce diabetic complications. This is a likely hypothesis because age-dependent hematopoietic dysfunction results mainly from abnormalities in osteoblastic niche cells [15]. Mayack et al. reported that young mice-derived long-term reconstituting HSCs (LT-HSC) co-cultured with aged mice-derived osteoblastic niche cells showed a reduced capacity for hematopoietic reconstitution compared with young mice-derived LT-HSCs co-cultured with young mice-derived osteoblastic niche cells [15]. Moreover, the exposure of normal LT-HSCs to aged osteoblastic niche cells is sufficient to worsen LT-HSCs function [15]. However, no investigations have been made into whether the exposure of abnormal LT-HSCs to normal osteoblastic niche cells can reverse the LT-HSC abnormality.

### SUMMARY OF THE INVENTION

In the present study, we used in vivo experiments to examine whether diabetes induces the abnormal expression of molecules, which are essential for maintaining quiescence and reconstitution of hematopoietic stem cells in the bone marrow niche. Using in vitro co-culture experiments, we examined whether exposure of normal LT-HSCs to diabetic osteoblastic niche cells induced the abnormal expression of molecules on LT-HSCs seen in in vivo experiments. We further examined whether the abnormal expression of molecules on diabetic LT-HSCs can be reversed following exposure to normal osteoblastic niche cells. Finally these results obtained from in vitro studies were confirmed by intra bone marrow-bone marrow transplantation with the replacement of osteoblastic niche cells and the effects on LT-HSCs were examined

Most HSCs are located at the trabecular bone surface of the bone marrow, and their interaction with osteoblastic cells through signaling and cell adhesion molecules appears to be essential to sustain quiescence and preserve the self-renewal of stem cells in normal hematopoiesis [19]. While long-term diabetes is known to impair the mobilization of hematopoietic progenitor cells [6, 8] or diminish the total number of HSCs in both human and mice, few studies have examined the interaction between HSCs and osteoblastic niche cells in diabetic conditions.

In the present study, we isolated LT-HSCs (c-kit⁺, Lin⁻, Sca-1⁺, CD34⁻, CD135⁻) and osteoblastic niche cells (Lin⁻, OPN⁺) from the bone marrow of mice with and without diabetes, and we examined the expression of molecules that regulate quiescence, anti-apoptosis and cell adhesion to maintain hematopoietic reconstitution.

FACS analysis showed that the number of osteoblastic niche cells and LT-HSCs was reduced and that of ST-HSCs and MPPs increased in diabetic mice. Because numbers of osteoblastic niche cells and LT-HSCs are correlated, depletion of the former may be caused by depletion of the latter. The observed increase in ST-HSCs and MPPs suggests that, in diabetes, osteoblastic niche cells failed to keep LT-HSCs quiescent so that their differentiation into ST-HSCs and MPPs was accelerated. Further evidence for this came from the observation that Ang-1 expression on osteoblastic niche cells and Tie2 expression on HSCs was reduced in diabetic mice. Ang-1 is produced mainly by osteoblastic niche cells, and its receptor tyrosine kinase, Tie2, is expressed on LT-HSCs. Tie2/Ang-1 signaling promotes tight adhesion of HSCs to osteoblastic niche cells and, under normal conditions, maintains both the quiescence and enhanced survival of HSCs [4].

Although the hematopoiesis activity of Lin⁻Sca-1⁺ c-kit⁺ cells was not changed between diabetic and nondiabetic mice as measured by colony forming activity in the present study, the phagocytotic activity of osteoclasts, those are derived from hematopoietic progenitors [20], was abnormally increased in diabetic mice. Hyperactivity of osteoclasts in diabetic state has been shown in previous study [21].

The present results also revealed that the expression of N-cadherin on LT-HSCs and the expression of β1-integrin on osteoblastic niche cells were reduced in diabetic mice. As N-cadherin-mediated adhesion mediates the slow cycling and quiescence of HSCs [4, 22, 23] and β1-integrin is essential for the interaction between bone marrow niche cells and HSCs and in regulating the initial self-renewing HSC division and survival [24], our finding suggests that HSCs failed to maintain quiescence, self-renewal and survival in diabetes.

The Wnt signal cascade is triggered upon binding to a co-receptor complex, including frizzled (Fzd) and LRP5/6 [25-28]. Wnt, Fzd and Dkk are included in the β-catenin phosphorylation complex, which leads to β-catenin degradation, and Dkk binds to and inactivates signaling from LRP5/6 receptors. Wnt/β-catenin signaling may play a crucial role in the maintenance of self-renewal activity in bone marrow. In the present study, we showed that both LRP6 expression on LT-HSCs and β-catenin expression on LH-HSCs were reduced in diabetic mice, suggesting that diabetes impairs the Wnt/β-catenin pathway resulting in an HSC reconstituting abnormality.

Chemokines and their receptors control HSCs behavior by regulating the migration, homing and release of HSCs within bone marrow. The importance of CXCL12/CXCR4 signaling was previously demonstrated by CXCL12^{-/-} and CXCR4^{-/-} mice [29, 30], which have a severe defect in their bone marrow myeloid progenitors. We show here that diabetes induced the depletion of CXCL12 expression by osteoblastic niche cells, suggesting an impaired interaction between osteoblastic niche cells and LT-HSCs in the bone marrow.

Our in vitro co-culture between LT-HSCs and osteoblastic niche cells under normal and high glucose concentration media aimed to mimic the diabetic and nondiabetic microenvironment in the bone marrow niche. The morphological feature of interaction between osteoblastic niche cells and LT-HSCs in co-culture experiments was shown to be similar to the in vivo localization of osteoblastic niche cells and LT-HSCs in the bone marrow sections. Furthermore the staining pattern was not changed between nondiabetic and diabetic mice, suggesting that surface phenotype of osteoblastic niche cells and LT-HSCs may hold in diabetic mice separately from their functional abnormality. In co-culture experiments osteoblastic niche cells derived from nondiabetic mice could adhere to the culture plate for seven days in normoglycemic but not hyperglycemic conditions. The reverse was true for osteoblastic niche cells derived from diabetic mice, suggesting that such cells adapt to higher glucose concentrations. The survival frequency of LT-HSCs paralleled the existence of osteoblastic niche cells. Therefore, our co-culture experiments provide an adequate microenvironment for keeping LT-HSCs in contact with osteoblastic niche cells and maintaining stemness for seven days. Attempts have previously been made to reconstitute a bone marrow niche using a 3D [31, 32] or low oxygen tension [33] co-culture system. However, as shown here, a conventional 2D co-culture appears to be sufficient.

The co-culture experiments showed the same abnormality in the LT-HSC expression of N-cadherin, β-catenin and Tie2 seen in in vivo diabetic mice. Different co-culture combinations between LT-HSCs and osteoblastic niche cells provided important findings; nondiabetic LT-HSCs co-cultured with diabetic osteoblastic niche cells led to reduced expression of these molecules, perhaps through impairment in self-renewal, survival and quiescence of HSCs. Most interestingly, the in vitro exposure of diabetic LT-HSCs to nondiabetic osteoblastic niche cells successfully reversed these abnormalities in expression. Although it is known that diabetes induces impaired hematopoietic stem/progenitor cell mobilization and repopulation by altering bone marrow niche functions [3, 8], no previous studies have shown the effects of the replacement of bone marrow niche cells on their partner hematopoietic stem cells. Our in vivo intra bone marrow-bone marrow transplantation experiments revealed that the replacement of osteoblastic niche cells could successfully reverse the abnormality in LT-HSCs caused by diabetes.

In summary we demonstrated that diabetes induces impairment in the expression of molecules on both LT-HSCs and osteoblastic niche cells, which are essential for maintaining quiescence and reconstitution of hematopoietic stem cells in the bone marrow niche. Normal LT-HSCs displayed abnormal expression of these molecules when exposed to diabetic osteoblastic niche cells, but this abnormality could be reversed after exposure to nondiabetic osteoblastic niche cells. Athough it is not known at present how diabetic metabolism impairs the osteoblastic niche cells, the present results give important perspectives for the treatment of diabetes-induced HSC abnormalities by the replacement of bone marrow niche cells.

### DESCRIPTION OF THE DRAWINGS

FIGS. 1A and 1B. Isolation of osteoblastic niche cells, LT-HSCs, ST-HSCs and MPPs from the bone marrow. FIG. 1A: Representative FACS plot for isolation of LT-HSCs (1) (Lin− Sca-1+c-Kit+CD135-CD34−), ST-HSCs (2) (Lin-Sca-1+ c-Kit+CD135− CD34+), MPPs (3) (Lin− Sca-1+ c-Kit+ CD135+ CD34+) and osteoblastic niche cells (Lin− OPN+). FIG. 1B: Frequency of osteoblastic niche cells, LT-HSCs, ST-HSCs and MPPs in Lin− bone marrow cells from diabetic and nondiabetic mice. Values are means±SD (* p<0.05 compared to nondiabetic mice, n=8).

FIGS. 2A-2E. Comparison of various molecules expressed on osteoblastic niche cells and LT-HSCs derived from diabetic and nondiabetic mice. FIG. 2A: Expressions of adhesion molecules N-cadherin, β-catenin and β1-integrin on osteoblastic niche cells and LT-HSCs. FIG. 2B: Expressions of Ang-1 and its ligand Tie2. FIG. 2C: Expressions of CXCL12 and its receptor CXCR4. FIG. 2D: Expressions of proteins related to Wnt/β-catenin signaling pathways. FIG. 2E: Expressions of TPO and its receptor MPL. Values are means±SD (*p<0.05 compared to nondiabetic mice, n=6−8).

FIG. 3 Summary of the expression of various molecules on the osteoblastic niche cell and LT-HSC derived from diabetic and nondiabetic mice. Small caption and dotted lines in lower panel represent the decrease in expression.

FIGS. 4A and 4B. In vitro co-culture between osteoblastic niche cells and LT-HSCs. FIG. 4A: Phase-contrast microscopic observation of osteoblastic niche cells (arrows) and LT-HSCs (arrowheads) seen at day 7 of co-culture. LT-HSCs (arrowheads) are attached to the osteoblastic niche cell. Bar=10 □m FIG. 4B: Comparison of the number of osteoblastic niche cells and that of LT-HSCs, derived from diabetic and nondiabetic mice co-cultured for 7 days at normal (100 mg/dl) and high glucose (450 mg/dl) condition. Values are means±SD (*p<0.05, †<0.05 compared to other groups, n=6−8).

FIGS. 5A-5C. Expression of molecules on LT-HSCs co-cultured with osteoblastic niche cells in different combinations. mRNA expression of N-cadherin (FIG. 5A), β-catenin (FIG. 5B) and Tie2 (FIG. 5C) on nondiabetic (a, c) or diabetic (b, d, e) LT-HSCs co-cultured with nondiabetic (a, d, e) or diabetic (b, c) osteoblastic niche cells in normal (100 mg/dl) and high (450 mg/dl) glucose condition. Values are means±SD (*p<0.05, n=6−8).

FIGS. 6A-6C. Isolation of cells from bone marrow. FIG. 6A: Representative FACS plot for isolation of LT-HSCs (1), ST-HSCs (2), MPPs (3) and osteoblastic niche cells. FIG. 6B: Cell frequency in Lin− bone marrow cells. FIG. 6C: Localization of OPN+ osteoblastic niche cell (arrow, red) and CD150+ LT-HSC (arrowhead, green) in the bone marrow. Values are means±SD for n=8, *p<0.05 compared with nondiabetic mice.

FIGS. 7A and 7B. Activity of Lin− c-kit+ Sca-1+ cells. FIG. 7A-left: The frequency of Lin− c-kit+ Sca-1+ cells in mononuclear cells by FACS. FIG. 7A-right: HSC colony forming activity. FIG. 7B: The TRAPSb activity. Values are means±SD for n=4−6, *p<0.05 compared with nondiabetic mice.

FIGS. 8A-8E. Comparison of molecule expression. FIG. 8A: Expression of N-cadherin (left axis), β-catenin (left axis) and β1-integrin (right axis) on osteoblastic niche cells and LT-HSCs. FIG. 8B: Expression of Ang-1 and Tie2. FIG. 8C: Expression of CXCL12 and CXCR4. FIG. 8D: Expression of proteins related to Wnt/β-catenin signaling pathways. FIG. 8E: Expression of TPO and MPL. Values are means±SD for n=6−8, *p<0.05 compared with nondiabetic mice.

FIG. 9 Summary of the expression of molecules on osteoblastic niche cells and LT-HSCs derived from diabetic and nondiabetic mice.

FIGS. 10A and 10B. In vitro co-culture. FIG. 10A-a: Phase-contrast microscopy of osteoblastic niche cells (arrows) and LT-HSCs (arrowheads) on day 7. Scale bar=10 □m. FIG. 10A-b: Qtracker™ 525 (green) is detected in the cytoplasm of LT-HSCs (arrow heads) and OPN-positive reaction (red) is seen in the cytoplasm of osteoblastic niche cells (arrows). Scale bar=10 □m. FIG. 10B: Numerical comparison of osteoblastic niche cells and LT-HSCs derived from diabetic and nondiabetic mice co-cultured at normal (100 mg/dl) and high glucose (450 mg/dl) conditions. Values are means±SD for n=6−8, *p<0.05 and ∀p<0.05 compared with other groups.

FIGS. 11A-11C. Expression of molecules on LT-HSCs co-cultured with osteoblastic niche cells. mRNA expression of N-cadherin (FIG. 11A), β-catenin (FIG. 11B) and Tie2 (FIG. 11C) on nondiabetic (a, c) or diabetic (b, d, e) LT-HSCs co-cultured with nondiabetic (a, d, e) or diabetic (b, c) osteoblastic niche cells in normal (100 mg/dl) and high (450 mg/dl) glucose conditions. Values are means±SD for n=6−8, *p<0.05 compared with other groups.

FIG. 12. Expression of Tie2 on LT-HSCs in IBM-BMT mice. The mixture of nondiabetic (a, c) or diabetic (b, d) OPN+ cells and nondiabetic (a, d) or diabetic (b, c) OPN—cells were transplanted into left tibia. mRNA expression of Tie2 on LT-HSCs at 7 days after IBM-BMT. Values are means±SD for n=4−5, *p<0.05 compared with other groups.

### DETAILED DESCRIPTION OF THE INVENTION

This description of an embodiment, set out below to enable one to build and use an implementation of the invention, is not intended to limit the invention, but to serve as a particular example thereof. Those skilled in the art should appreciate that they may readily use the conception and specific embodiments disclosed as a basis for modifying or designing other methods and systems for carrying out the same purposes of the present invention.

The term “Osteoblastic niche cells”, as used herein, corresponds to a subset of osteoblasts supposed to function as a key component of the HSC niche which interacts with HSCs and controls their number. Osteoblastic niche cells can be isolated from bone marrow by such methods as magnetic cell separation and/or flow-cytometry based on surface-marker expression. Markers used for the separation of osteoblastic niche cells may be any one or a combination of those selected from a group including Wnt⁺, CXCL-12⁺, TPO⁺, Ang-1⁺, N-cadherin⁺, β3-catenin⁺, β1-integrin⁺, osteopontin⁺, CD45⁻, and Lin⁻, preferably osteopontin⁺ and Lin⁻. Bone marrow used for isolation of osteoblastic niche cells can be obtained by any conventional methods including crushing bone followed by digestion with trypsin and/or collagenase.

LT-HSCs can be isolated by any conventional methods including magnetic cell separation and/or flow-cytometry based on surface-marker expression.

Cells can be cultured any conventional ways including methods described herein.

One embodiment of the present invention provides a method of establishing in vitro co-culture of LT-HSCs and osteoblastic niche cells, comprising the steps of: isolating LT-HSCs; isolating osteoblastic niche cells; mixing said isolated LT-HSCs and said isolated osteoblastic niche cells while transferring them to a tissue culture dish; culturing the cell mixture until LT-HSCs and osteoblastic niche cells attach together on the culture dish to form 2-dimensional co-culture system; and further culturing said 2-dimensional co-culture system. The culture is maintained more than 3 days, preferably at least 5 days, more preferably up to 7 days, or at least 7 days or more, while preserving both viability of the osteoblastic niche cells and the immature phenotype of the LT-HSCs. The phenotype of the osteoblastic niche cells is; Wnt⁺, CXCL-12⁺, TPO⁺, Ang-1⁺, N-cadherin⁺, β-catenin⁺, β1-integrin⁺, osteopontin⁺, CD45⁻, and Lin⁻. The LT-HSCs may be isolated from bone marrow or blood of mammal, preferably mouse, rat or human. The osteoblastic niche cells may be isolated from bone marrow of mammal, preferably mouse, rat or human.

In another embodiment, the present invention provides an in vitro co-culture system of LT-HSCs and osteoblastic niche cells obtained by using the methods described above.

In some embodiments, the present invention provides a method of screening people suffering from a disorder caused by dysfunction of osteoblastic niche cells, comprising: (a) isolating LT-HSCs from the subject for which presence of disorder is to be determined, then (b) detecting in said LT-HSCs differential expression of at least one gene selected from the group consisting of: N-cadherin, LRP6, β-catenin, and Tie2, wherein downregulation of expression of said at least one gene indicates presence of said disorder.

In some embodiments, the present invention also provides a method of screening people suffering from a disorder caused by dysfunction of osteoblastic niche cells, comprising: (a) isolating osteoblastic niche cells from the subject for which presence of disorder is to be determined, then (b) detecting in said osteoblastic niche cells differential expression of at least one gene selected from the group consisting of: CXCL-12, Ang-1, β-catenin⁺ and β1-integrin, wherein downregulation of expression of said at least one gene indicates presence of said disorder.

In certain further embodiments, the present invention provides a method of screening a candidate substance for treatment of a disorder caused by dysfunction of osteoblastic niche cells, comprising: (a) providing 2-dimensional co-culture system of LT-HSCs and osteoblastic niche cells according to the method of claim 1, then (b) exposing said co-culture system to a test substance; and then (c) detecting in LT-HSCs of the culture system differential expression of at least one gene selected from the group consisting of: N-cadherin, LRP6, β-catenin, and Tie2, wherein upregulation of expression of said at least one gene indicates that the reagent is a candidate reagent for said disorder.

In still further aspects, the present invention provides a method of screening a candidate substance for treatment of a disorder caused by dysfunction of osteoblastic niche cells, comprising: (a) providing 2-dimensional co-culture system of LT-HSCs and osteoblastic niche cells according to the method of claim 1, then (b) exposing said co-culture system to a test substance; and then (c) detecting in osteoblastic niche cells of the culture system differential expression of at least one gene selected from the group consisting of: CXCL-12, Ang-1, β-catenin⁺ and β1-integrin, wherein upregulation of expression of said at least one gene indicates that the reagent is a candidate reagent for the disorder.

In some embodiments of the present invention, there is provided a method of treating a patient suffering from a disorder caused by dysfunction of osteoblastic niche cells, comprising: (a) providing osteoblastic niche cells of which expression of at least one gene selected from the group consisting of: CXCL-12, Ang-1, β-catenin⁺ and β1-integrin, is within normal levels, and then (b) administrating an effective amount of said osteoblastic niche cells.

Expression within “normal” levels, as used herein, means between half and twice of the average mRNA and/or protein expression level of the corresponding gene for healthy people.

In certain aspects, osteoblastic niche cells to be administered are allogeneic to the patient. Such osteoblastic niche cells can be checked for CXCL-12, Ang-1, β-catenin⁺ or β1-integrin expression to be within normal levels.

In another aspect, osteoblastic niche cells to be administered are collected from the same patient. Such osteoblastic niche cells can be co-cultured as above with LT-HSCs which are collected from a healthy donor before administration.

In some embodiments of the present invention, there is also provided a method of treating a patient suffering from a disorder caused by dysfunction of osteoblastic niche cells, comprising: (a) providing LT-HSC of which expression of at least one gene selected from the group consisting of: N-cadherin, LRP6, β-catenin, and Tie2, is within normal levels, and then (b) administrating an effective amount of said LT-HSC. Such LT-HSC cells can be collected from the patient. The LT-HSC cells collected from the patient can be co-cultured before administration as above with osteoblastic niche cells which are collected from a healthy donor.

Administration of cells herein can be through topical application. The topical application may comprise injection of said cells into bone marrow. Administration of cells herein can also be systemic.

Optionally, administration of cells herein can be performed after the patient is undergone radiotherapy or chemotherapy with carboplatin, etoposide, cyclophosphamide, doxorubicin, or topotecan.

According to embodiments of the present invention, there may be provided a method involving treating a patient suffering from a disorder caused by dysfunction of osteoblastic niche cells, comprising administering a factor selected from a group including Ang-1, β1 integrin, β-catenin⁺ and CXCL-12, and their analogues.

There is also provided in the present invention a therapeutic composition for treating a patient suffering from a disorder caused by dysfunction of osteoblastic niche cells, which comprises osteoblastic niche cells as an active ingredient.

The disorder caused by dysfunction of osteoblastic niche cells, as described herein, may be any selected from groups of disorders including: autoimmune diseases including nephrosis, glomerulonephritis, type 1 and type 2 diabetes mellitus and their complications, vitiligo, Crohn's disease, ulcerative colitis, Behcet's syndrome, and psoriasis; collagen diseases including chronic rheumatism, scleroderma, systemic lupus erythematosus, and dermatomyositis; blood disorders including leukemia, aplastic anemia, and myelodysplastic syndrome; degenerative diseases including Alzheimer's disease, multiple sclerosis, amyotrophic lateral sclerosis, and osteoporosis; chronic inflammations including arteriosclerosis, emphysema, chronic obstructive pulmonary disease, hepatic cirrhosis, and atopic dermatitis; and malignant tumors.

The effect and safety of the treating agent of the present invention is illustrated by the following experiments.

### Mice

C57BL/6 male mice were purchased from the Sankyo Laboratory (Tokyo, Japan). Diabetes was induced by a single intraperitoneal (ip) injection of 150 mg/kg streptozotocin (STZ) dissolved in citrate buffer (pH 4.5) to 8- to 10-week-old mice. Four weeks after the injection blood glucose levels were assayed, and mice those blood glucose concentration of >400mg/dl were used in present study. Nondiabetic control mice were injected with 100 μl vehicle.

### Flow Cytometry

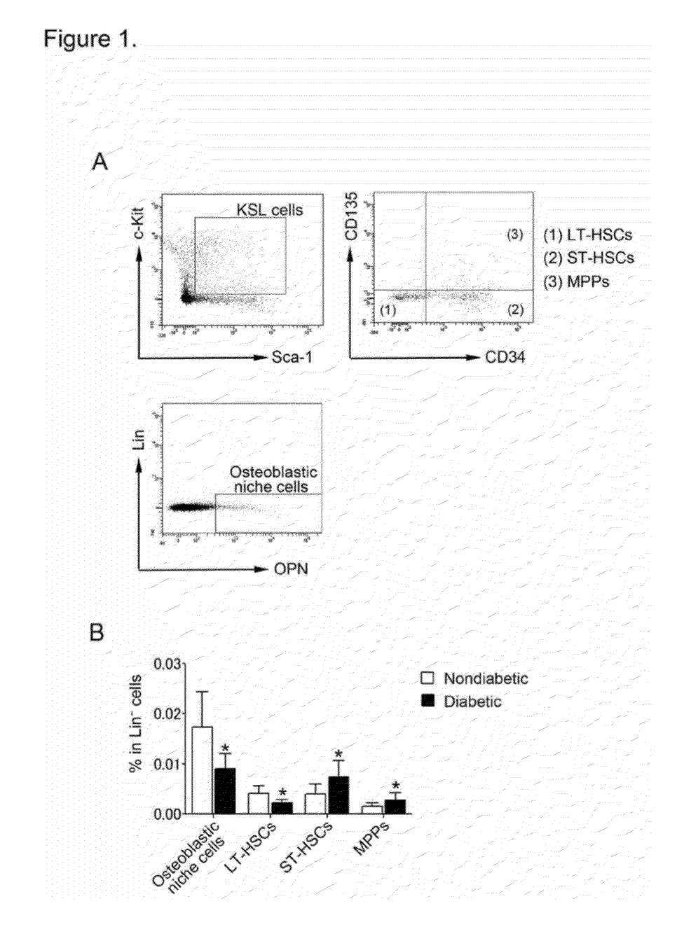

LT-HSCs and Osteoblastic niche cells were sorted as Lin⁻Sca-1⁺c-Kit⁺CD135⁻CD34⁻ cells and Lin⁻OPN⁺ cells, respectively (Mayack SR et al 2010). Femora and tibiae of 4 mice treated with vehicle or STZ were crushed and digested at 37° C. for 1 hour with Hanks' buffered saline including 0.25% trypsin, 1 mM EDTA (25200, Life Technologies, NY), and 0.1% collagenase (034-10533. WAKO, Japan). Cells were washed with phosphate buffered saline (pH7.5) containing 2% FCS and filtrated through a 40 μm cell strainer filter (BD Bioscience). Erythrocytes were lysed by ammonium chloride potassium (ACK) lysis buffer (Qiagen). Cell suspension was incubate with a biotinylated lineage cocktail (anti-CD5, anti-CD45R (B220), anti-CD11b, anti-Gr-1 anti-Ly-6G/C, and anti-Ter-119, 130-090-858, Miltenyi Biotec Inc., CA) followed by anti-biotin MicroBeads (130-090-485, Miltenyi Biotec Inc., CA), and Lin⁻ cells were isolated with the MACS column and separator (Miltenyi Biotec Inc., CA). Obtained Lin⁻ cells were stained with anti-c-Kit-APC-Cy7 (2B8, BD Biosciences), anti-Sca-1-PE-Cy7 (D7, Biolegend), anti-CD34-Alexa Flour 700 (RAM34, BD Biosciences), anti-CD135-PE (A2F10, Biolegend), and anti-OPN (Immuno-Biological Laboratories). For secondary antibody for OPN, anti-rabbit IgG conjugated APC (R&D Systems) was used. DAPI dye was used to exclude dead cells (DOJINDO). Lin⁻ Sca-1⁺c-Kit⁺ (KSL) cells were sorted first, and KSL cells were subsequently isolated by CD135 and CD34 antibodies (FIG. 1Aa). CD135⁻CD34⁻ cells were identified as LT-HSCs, CD135⁻CD34⁺ cells were identified as short-term reconstituting HSCs (ST-HSCs) and CD 135⁺CD34⁺ cells were identified as multipotent hematopoietic progenitors (MPPs) (FIG. 1A). Lin⁻OPN⁺ cells were identified as osteoblastic niche cells (FIG. 1A).

For sorting of LT-HSCs and osteoblastic niche cells after co-culture experiments, culture medium was discarded and adherent cells were removed by Hanks' buffered saline including 0.25% trypsin, 1 mM EDTA (25200, Life Technologies, NY) for 15 minutes and processed for FACS as mentioned above.

### Co-Culture of Osteoblastic Niche Cells and LT-HSCs

To investigate the interactions between LT-HSCs and osteoblastic niche cells, sorted LT-HSCs (6×10³ cells) and osteoblastic niche cells (6×10⁴ cells) were co-cultured. The medium used was normal glucose (100 mg/dl) or high glucose (450 mg/dl) containing DMEM supplemented with 10% FBS, 1% penicillin, 150 ng/mL FLT3-ligand, 150 ng/ml SCF and 150 ng/ml TPO. Osteoblastic niche cells and LT-HSCs were seeded at the same time in 16-well chamber slide (Thermo Scientific, USA) at 37° C. in 5% CO₂/air for 7 days. At day3, culture medium was changed. Morphology of the co-cultured cells was observed under phase-contrast microscopy.

### Quantitative RT-PCR Analysis

To examine the mRNA expression of various proteins, total RNA was extracted using RNeasy Plus Micro Kit (Qiagen), and cDNA was synthesized using the SuperScript First-Strand Systhesis System for RT-PCR (Invitrogen). Quantitative RT-PCR for N-cadherin, β-catenin, integrin-β, Tie-2, CXCR4, Frizzled (Fzd) receptor4, Fzd7, lipoprotein receptor-related protein 5 (LRP5), LRP6, thrombopoietin receptor (MPL), angiopoietin-1 (Ang-1), CXCL12, wingless-type MMTV integration site family, member 10b (Wnt10b), dickkopf-1 (Dkk1), and thrombopoietin (TPO) was performed on the ABI prism 7500 Sequence Detection System (Applied Biosystems, Foster City, Calif.) with Power SYBAR GREEN PCR Master Mix (4367659; Applied Biosystems, Foster City, Calif.). Relative mRNA expression was quantified by the 2-ACT method. Primer sequences are shown in Supplementary Table 1.

### Statistical Analysis

Differences between groups were determined using a two-tailed Student's t-test and one-way ANOVA followed by a Tukey's Multiple Comparison Test. Differences were considered significant at p <0.05.

### EXAMPLES

Animal Studies

C57BL/6 male mice were purchased from the Sankyo Laboratory (Tokyo, Japan). Diabetes was induced by a single intraperitoneal (ip) injection of 150 mg/kg STZ dissolved in citrate buffer (pH 4.5) to 8- to 10-week-old mice. Four weeks after the injection, blood glucose levels were assayed and mice with blood glucose concentrations >400mg/dl were used in the present study. Nondiabetic control mice were injected with 100 μl vehicle. Animal experiments were approved by the animal use committee of Sapporo Medical University, Sapporo, Hokkaido, Japan.

Flow Cytometry

LT-HSCs and osteoblastic niche cells were sorted as Lin⁻Sca-1⁺ c-Kit⁺ CD135⁻ CD34⁻ cells and Lin⁻OPN⁺ cells, respectively [15]. Femora and tibiae of four mice treated with vehicle or STZ were crushed and digested at 37° C. for 1 h with Hanks' buffered saline including 0.25% trypsin, 1 mM EDTA (25200, Life Technologies, Grand Island, N.Y.), and 0.1% collagenase (034-10533. WAKO, Osaka, Japan). Cells were washed with phosphate buffered saline (pH 7.5) containing 2% fetal calf serum (FCS) and filtered through a 40 μm cell strainer filter (BD Biosciences, Franklin Lakes, N.J.). Erythrocytes were lysed by ammonium chloride potassium (ACK) lysis buffer (Qiagen, Tokyo, Japan). Cell suspensions were incubated with a biotinylated lineage cocktail (anti-CD5, anti-CD45R (B220), anti-CD11b, anti-Gr-1 anti-Ly-6G/C, and anti-Ter-119, 130-090-858, Miltenyi Biotec Inc., Auburn, Calif.) followed by anti-biotin MicroBeads (130-090-485, Miltenyi Biotec Inc.). Lin⁻ cells were isolated with the MACS column and separator (Miltenyi Biotec Inc.) and stained with anti-c-Kit-APC-Cy7 (2B8, BD Biosciences), anti-Sca-1-PE-Cy7 (D7, Biolegend, San Diego, Calif.) anti-CD34-Alexa Flour 700 (RAM34, BD Biosciences), anti-CD135-PE (A2F10, Biolegend), and anti-OPN (18621, Immuno-Biological Laboratories, Gunma, Japan). Anti-rabbit IgG-conjugated APC (R&D Systems, Minneapolis, Minn.) was used as the anti-OPN secondary antibody.

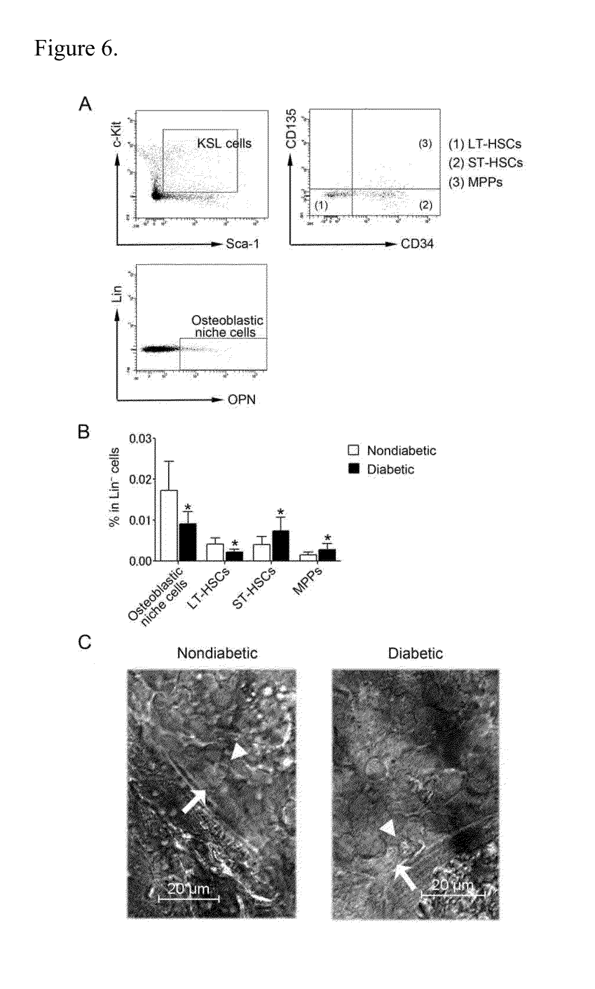

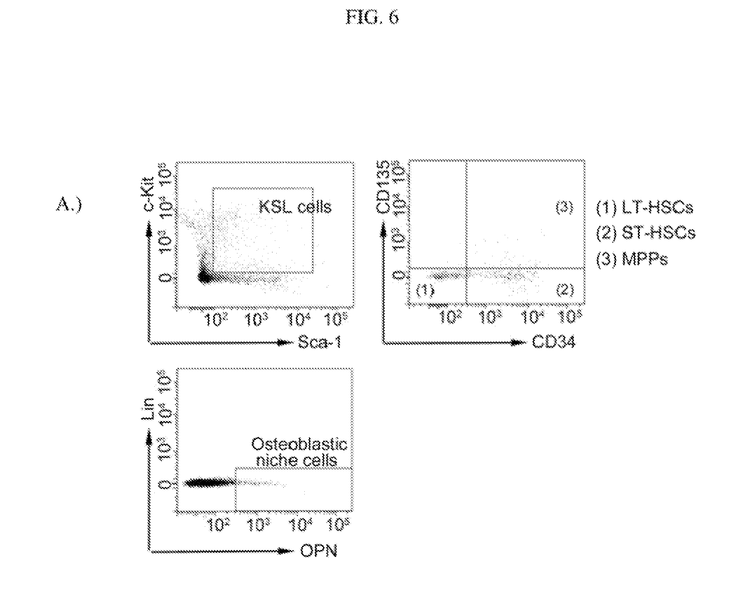

6-Diamidino-2-phenylindole dihydrochloride solution (DAPI, D523, DOJINDO, Kumamoto, Japan) was used to exclude dead cells. Lin⁻Sca-1⁺c-Kit⁺ (KSL) cells were sorted first, and KSL cells were subsequently isolated by CD135 and CD34 antibodies (FIG. 6A). CD135⁻ CD34⁻ cells were identified as LT-HSCs, CD 135⁻ CD34⁺ cells as short-term reconstituting HSCs (ST-HSCs) and CD135⁺ CD34⁺ cells as multipotent hematopoietic progenitors (MPPs) (FIG. 6A). Lin⁻ OPN⁺ cells were identified as osteoblastic niche cells (FIG. 6A).

After co-culture experiments, culture medium was discarded and adherent cells were removed by Hanks' buffered saline including 0.25% trypsin and 1 mM EDTA (25200, Life Technologies) for 15 mM Sorting of LT-HSCs and osteoblastic niche cells was carried out by FACS as mentioned above.

Osteoblastic Niche Cells and LT-HSCs Co-culture

To investigate the interactions between LT-HSCs and osteoblastic niche cells, sorted LT-HSCs (6x10³ cells) and osteoblastic niche cells (6x10⁴ cells) were co-cultured. The medium used was normal glucose (100 mg/dl) or high glucose (450 mg/dl) containing DMEM supplemented with 10% FBS, 1% penicillin, 150 ng/mL Fms-like tyrosine kinase 3 (FLT3) ligand, 150 ng/ml SCF and 150 ng/ml TPO. Osteoblastic niche cells and LT-HSCs were seeded at the same time in 16-well chamber slides (Thermo Scientific, Barrington, Ill.) at 37° C. in 5% CO₂/air for seven days. On day 3, the culture medium was changed. Co-cultured cell morphology was observed under phase-contrast microscopy.

HSCs Colony Forming Assay

To examine the repopulation activity of hematopoietic cells, Lin⁻ c-kit⁺ Sca-1⁺ cells were sorted from femora and tibiae of vehicle and STZ-injected mice with FACS and cultured in CytoSelect Methylcellulose Medium (CBA-320, Cell Biolabs Inc., CA) containing 10% FBS, 500-ng/ml stem cell factor, 100-ng/ml IL-3, 100-ng/ml granulocyte macrophage colony-stimulating factor, and 30-ng/ml erythropoietin at 5% CO₂/air and 37° C. for 7 days. Colony formation was measured with CyQuant GR dye solution (CBA-320, Cell Biolabs Inc., CA). The number of sorted Lin⁻ c-kit⁺ Sca-1⁺ cells was counted with FACS.

Serum TRAP5b Assay

To compare the phagocytotic activity of osteoclasts in vehicle- and STZ-injected mice, blood was collected and centrifuge at 300 g for 10 min, and serum tartrate-resistant acid phosphatase (TRAP) 5b was measured using mice TRAP assay kit (SB-TR103, Immunodiagnostic Systems Ltd., AZ).

Immunofluorescence Staining for Bone Marrow Sections

Mice were anesthetized with pentobarbital and perfused with 4% paraformaldehyde in phosphate buffer. Isolated bones were decalcified with decalcifying solution B (041-22031, WAKO, Osaka, Japan), and cryostat-cut sections were prepared using adhesive film (16). Sections were blocked with 2% goat serum, incubated in rabbit anti-mouse OPN (ab8448, abcam, MA) and PE conjugated rat anti-mouse CD150 antibodies, a maker of LT-HSC [17], and further incubated secondary antibody Texas Red-labeled goat anti rabbit IgG (TI-1000, Vector, CA).

Immunofluorescence Staining for Cultured Cells

Morphological analysis for co-culture cells labeled with different fluorescence color was performed. Isolated LT-HSCs were incubated at 37° C. for 1 hour with the culture medium containing Qtracker™ 525 (Invitrogen), and they were co-cultured with isolated osteoblastic niche cells at 37° C. in 5%CO2/air for 1 week. At day 7 co-cultured cells were fixed with 4% paraformaldehyde in PBS, incubated with 2% BSA in PBS, and stained with anti-OPN (1:50, Immuno-Biological Laboratories) in PBS containing 0.25% Triton X-100. For secondary antibody to OPN, anti-rabbit IgG conjugated Cy5 (1:500, ab6564, Abcam, Cambridge, UK) was used. Nuclei were stained with DAPI. Fluorescence images were obtained under a confocal laser microscope (A1; Nikon, Tokyo, Japan).

Quantitative RT-PCR Analysis

To examine the mRNA expression of various proteins, total RNA was extracted using the RNeasy Plus Micro Kit (Qiagen), and cDNA was synthesized using the SuperScript First-Strand Systhesis System for RT-PCR (Invitrogen, Grand Island, N.Y.). Quantitative RT-PCR for N-cadherin, β-catenin, β1-integrin, Tie2, CXCR4, Frizzled (Fzd) receptor4, Fzd7, lipoprotein receptor-related protein 5 (LRP5), LRP6, thrombopoietin receptor (MPL), angiopoietin-1 (Ang-1), CXCL12, wingless-type MMTV integration site family, member 10b (Wnt10b), dickkopf-1 (Dkk1), and TPO was performed on the ABI prism 7500 Sequence Detection System (Applied Biosystems, Foster City, Calif.) with Power SYBAR GREEN PCR Master Mix (4367659; Applied Biosystems). Relative mRNA expression was quantified by the 2-ΔCT method. Primer sequences for PCR are shown in Table 1.

TABLE 1

| | |
|---|---|
| N-cadherin F | 5′-AATCAGACGGCTAGACGAGAGG-3′ |
| N-cadherin R | 5′-TCAGCAGATTTAAGGCCCTCAT-3′ |
| β-catenin F | 5′-ACGCGTGTGAGAAAGAATACAGAC-3′ |
| β-catenin R | 5′-GCTTGAGTTTGGTTCTGGGC-3′ |
| β1-integrin F | 5′-TGGAAAATTCTGCGAGTGTGAT-3′ |
| β1-integrin R | 5′-TGCCAGTGTAATTGGGATAGCA-3′ |
| Wnt10b F | 5′-GCGAAGGATAATAGCAGGCAT-3′ |
| Wnt10b R | 5′-TTGTCACCCGAGGTCCCATA-3′ |
| Dkk1 F | 5′-CAAAAATGTATCACACCAAAGGACAA-3′ |
| Dkk1 R | 5′-TGCTTGGTACACACTTGACCT-TCTT-3′ |
| Fzd4 F | 5′-GTGGATGCCGATGAACTGAC-3′ |
| Fzd4 R | 5′-ACAGCGTTCCAATCACCAAA-3′ |
| Fzd7 F | 5′-TATCGCCTACAACCAGACCATC-3′ |
| Fzd7 R | 5′-GGGTGCGTACATAGAGCATAAGA-3′ |
| LRP5 F | 5′-CCCACTCACGGGTGTCAAA-3′ |
| LRP5 R | 5′-TGCTCCACTGAGCTCCCATT-3′ |
| LRP6 F | 5′-TGGCTTGGCGGTGTGAT-3′ |
| LRP6 R | 5′-CACACGGGACAATTGAGTTCA-3′ |
| Ang-1 F | 5′-CAGCACGAAGGATGCTGATAAC-3′ |
| Ang-1 R | 5′-TTGTCCCGCAGTGTAGAACATT-3′ |
| Tie2 F | 5′-AAGCATGCCCATCTGGTTAC-3′ |
| Tie2 R | 5′-GCCTGCCTTCTTTCTCACAC-3′ |
| CXCL12 F | 5′-GCTCTGCATCAGTGACGGTA-3′ |
| CXCL12 R | 5′-TAATTACGGGTCAATGCACA-3′ |
| CXCR4 F | 5′-CTTTGTCATCACACTCC-CCTT-3′ |
| CXCR4 R | 5′-GCCCACATAGACTGCCT-TTTC-3′ |
| TPO F | 5′-CCAGACGGAACAGAGCAAG-3′ |
| TPO R | 5′-CTGTCCTCGTGCTGCCAT-3′ |
| MPL F | 5′-CCTGCACTGGAGGGAGGTCT-3′ |
| MPL R | 5′-GGCTCCAGCACCTTCCAGTC-3′ |
| GAPDH F | 5′-CTACAGCAACAGGGTGGTGGAC-3′ |
| GAPDH R | 5′-GGATAGGGCCTCTCTTGCTCAG-3′ |

Intra Bone Marrow-Bone Marrow Transplantation

To evaluate the interaction of LT-HSC and osteoblastic niche cells in vivo, OPN⁺ cells and OPN⁻ cells were transplanted into bone marrow as follows. OPN⁺ and OPN⁻ cells were sorted from femora and tibiae of vehicle or STZ-injected mice with MACS. Mice were received irradiation (10 Gy) and transplanted with the mixture of 2×10⁶ OPN⁺ and 1.5×10⁴ OPN⁻ cells derived from either diabetic or nondiabetic mice via intra bone marrow-bone marrow transplantaion (IBM-BMT) one day after the irradiation according to the previously study (18). One week after the bone marrow transplantation, LT-HSCs were sorted by FACS as described above, and mRNA expression of Tie2 was examined.

Statistical Analysis

Differences between groups were determined using a two-tailed Student's t-test and one-way analysis of variance (ANOVA) followed by a Tukey's multiple comparison test. Differences were considered significant at p<0.05.

### Example 1

The percentage of osteoblastic niche cells, LT-HSCs, ST-HSCs and multipotent hematopoietic progenitors (MPPs) occupied in Lin⁻ bone marrow cells was compared between diabetic and nondiabetic mice. Percentage of osteoblastic niche cells, and that of LT-HSCs were significantly reduced in diabetic mice compared to nondiabetic mice (FIG. 1B). On the other hand, percentage of ST-HSCs and MPPs was significantly increased in diabetic mice compared to nondiabetic mice (FIG. 1B).

### Example 2

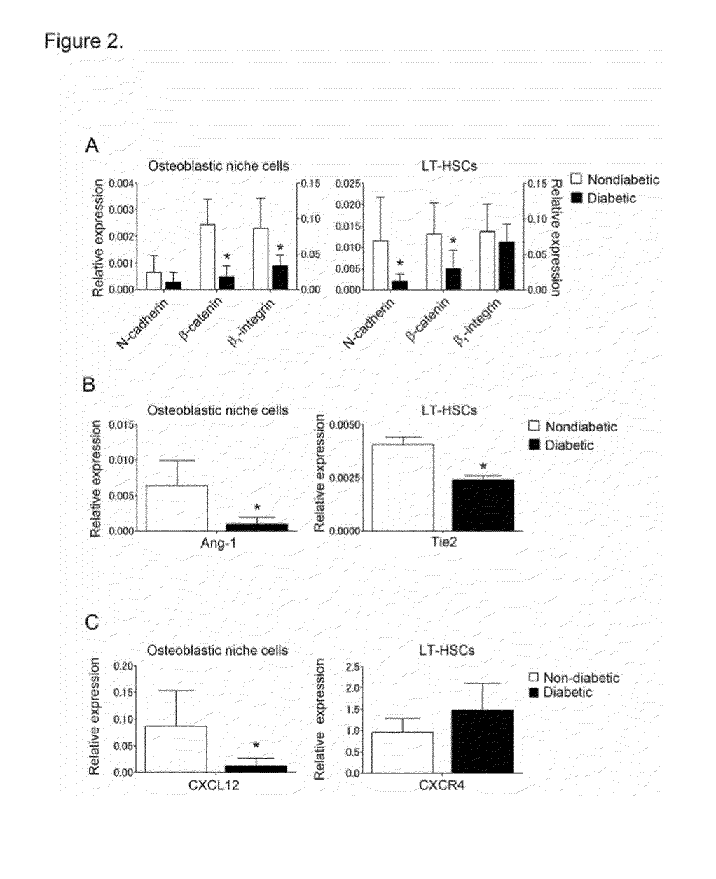

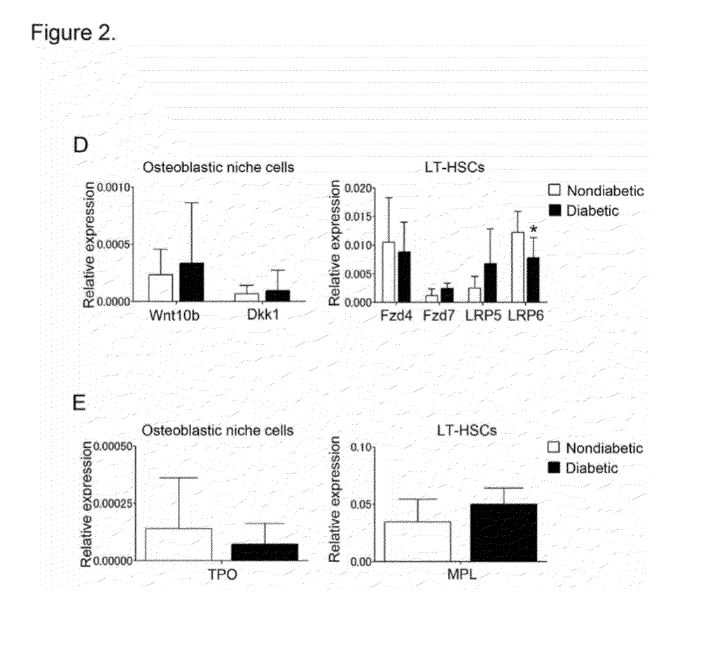

Expression of cell adhesion molecules such as N-cadherin, β1-catenin and β1-integrin on isolated osteoblastic niche cells, and LT-HSCs from diabetic mice was compared with those from nondiabetic mice. Results showed that the expression of β-catenin and β1-integrin on isolated osteoblastic niche cells, was significantly reduced in diabetic mice, however expression of N-cadherin on osteoblastic niche cells was not changed (FIG. 2A). The expression of N-cadherin and β-catenin on LT-HSCs was significantly reduced in diabetic mice compared to nondiabetic mice (FIG. 2A), while expression of β1-integrin on LT-HSCs was not changed between diabetic and nondiabetic mice (FIG. 2A).

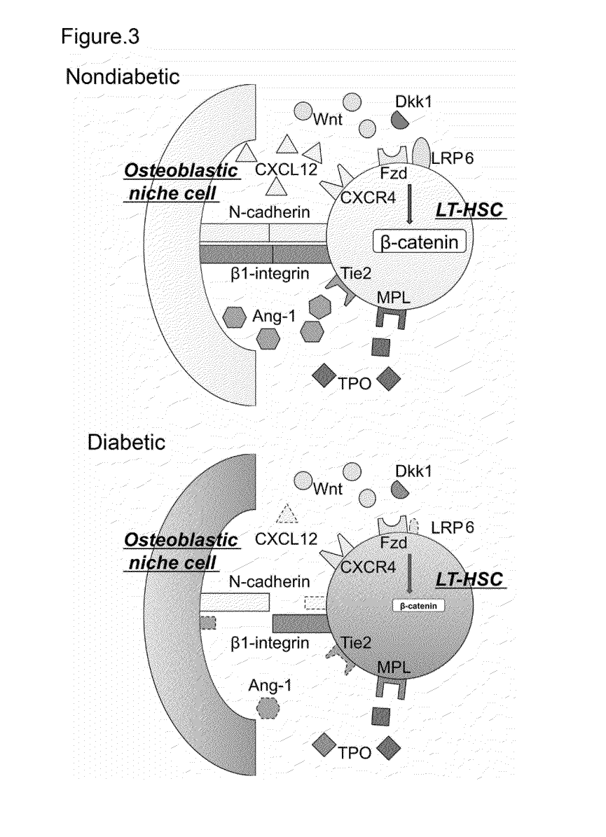

Ang-1 expression on osteoblastic niche cells from diabetic mice was significantly reduced compared to that from nondiabetic mice, and its ligand Tie2 expression on LT-HSCs was also significantly reduced in diabetic mice (FIG. 2B). CXCL12 expression on osteoblastic niche cells was significantly reduced in diabetic mice, but its receptor CXCR4 expression on LT-HSCs was not changed between diabetic and nondiabetic mice (FIG. 2C). In Wnt/β-catenin signaling pathways, Wnt 10b expression on osteoblastic niche cells was not changed, and receptors for Wnt family proteins such as Fzd4, Fzd7 and LRP5 on LT-HSCs were not changed, however another receptor LRP6 expression on LT-HSCs was reduced in diabetes (FIG. 2D). Dkkl expression on osteoblastic niche cells, was not changed between diabetic and nondiabetic mice (FIG. 2D). The expression of thrombopoietin (TPO) on osteoblastic niche cells, and expression of its receptor MPL on LT-HSCs were not changed between diabetic and nondiabetic mice (FIG. 2E). Summary of above results was shown in FIG. 3.

### Example 3

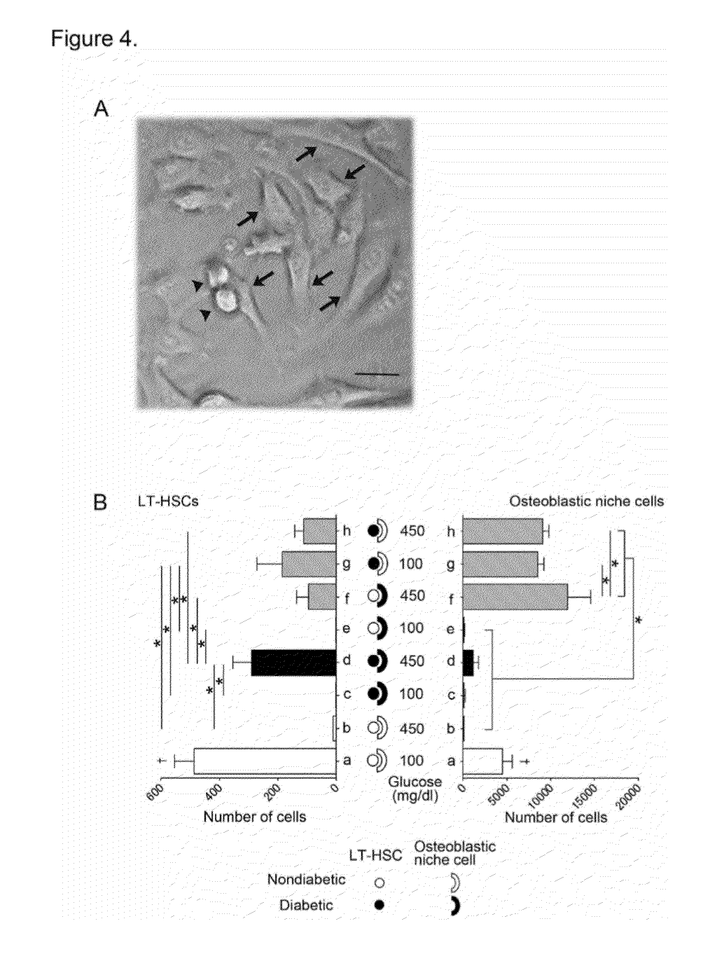

We performed in vitro co-culture experiments of LT-HSCs and osteoblastic niche cells, derived from diabetic and nondiabetic mice. After 7 days co-culture experiments, LT-HSCs were in contact with osteoblastic niche cells at the bottom of the culture plate (FIG. 4A), and these adherent cells were collected and subsequently isolated into LT-HSCs and osteoblastic niche cells by FACS. As shown in FIG. 4B, osteoblastic niche cells derived from nondiabetic mice can lodge on the plate in the medium with normal glucose concentration but not survive in the medium with high glucose concentration (FIG. 4Ba, b). On the other hand, osteoblastic niche cells derived from diabetic mice can lodge on the plate in the medium with high glucose concentration but not survive in normal glucose concentration (FIG. 4Bc, d, e, f). Parallel effects were found in the frequency of LT-HSCs. LT-HSCs cannot be maintained in the conditions which osteoblastic niche cells cannot lodge on the plate (FIG. 4Bb, c, e). However osteoblastic niche cells derived from nondiabetic mice co-cultured with LT-HSCs derived from diabetic mice can lodge on the plate even in high glucose condition (FIG. 4Bh).

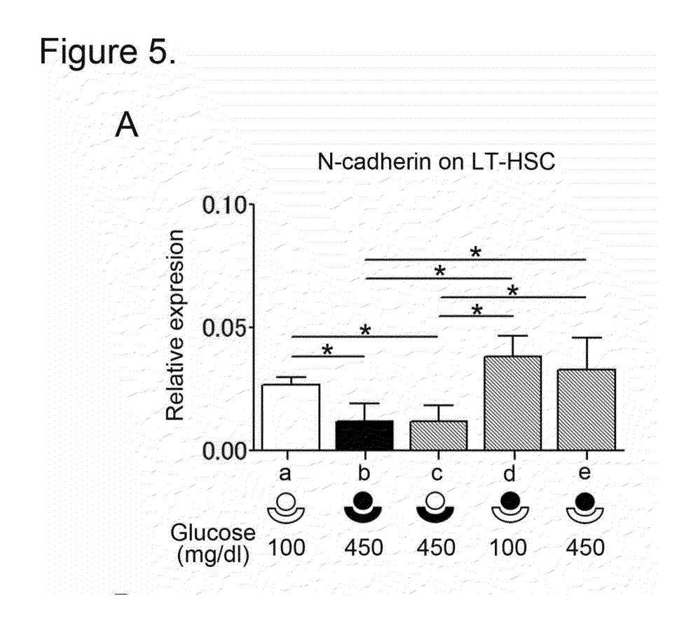

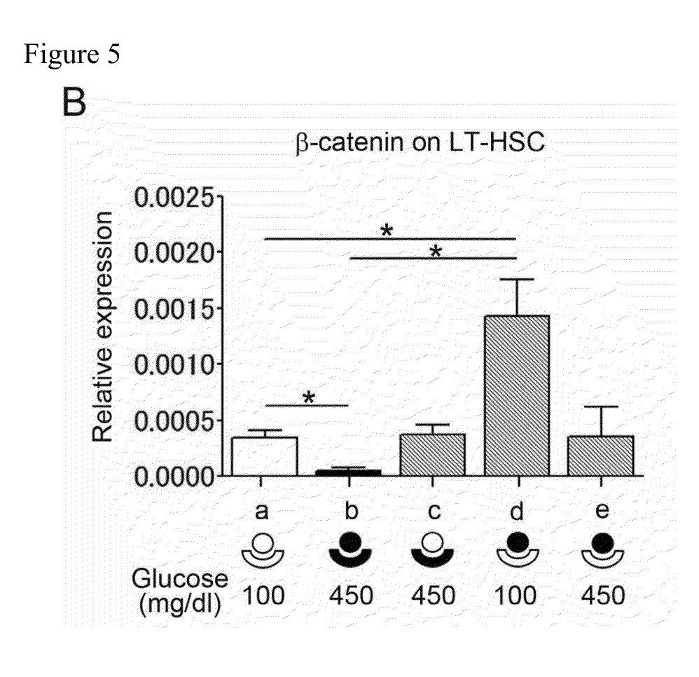

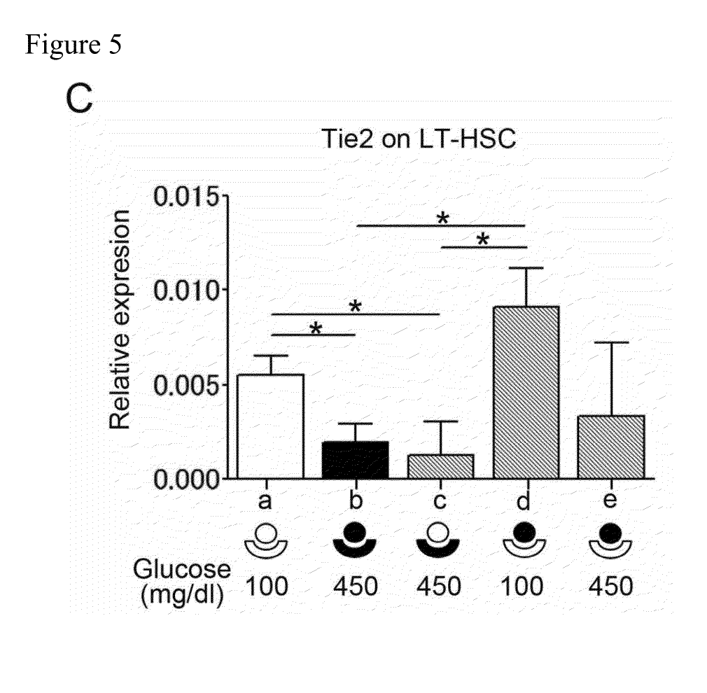

Expression of molecules on LT-HSCs derived from diabetic or nondiabetic mice was examined after co-cultured with osteoblastic niche cells derived from diabetic or nondiabetic mice in different glucose conditions, but only in cases cells were detected after 7 day co-culture as shown in FIG. 4B a, d, f, g and h. We chose N-cadherin, β-catenin and Tie2 on LT-HSC, because their expression was reduced in vivo diabetic model. N-cadherin expression on diabetic LT-HSCs exposed to diabetic osteoblastic niche cells was significantly reduced compared with that on nondiabetic LH-HSCs exposed to nondiabetic osteoblastic niche cells (FIG. 5Aa, b). N-cadherin expression on nondiabetic LT-HSCs exposed to diabetic osteoblastic niche cells in high glucose condition was significantly reduced compared those exposed to nondiabetic osteoblastic niche cells (FIG. 5Aa, c). Reduced expression of N-cadherin on diabetic LT-HSCs when exposed to diabetic osteoblastic niche cells (FIG. 5Ab) was reversed after exposure to nondiabetic osteoblastic niche cells in either normal or high glucose condition (FIG. 5Ad, e).

Similarly β-catenin expression on LT-HSCs was examined after in vitro co-culture with osteoblastic niche cells in different combinations. β-catenin expression on diabetic LT-HSCs exposed to diabetic osteoblastic niche cells was significantly reduced compared with that on nondiabetic LH-HSCs exposed to nondiabetic osteoblastic niche cells (FIG. 5Ba, b). Reduced expression of 3-catenin on diabetic LT-HSCs when exposed to diabetic osteoblastic niche cells (FIG. 5Bb) was reversed after exposure to nondiabetic osteoblastic niche cells in normal glucose condition (FIG. 5Bd).

Tie2 expression on diabetic LT-HSCs exposed to diabetic osteoblastic niche cells was significantly reduced compared with that on nondiabetic LH-HSCs exposed to nondiabetic osteoblastic niche cells (FIG. 5Ca, b). Tie2 expression on nondiabetic LT-HSCs co-cultured with diabetic osteoblastic niche cells in high glucose condition was significantly reduced compared to those exposed to nondiabetic osteoblastic niche cells (FIG. 5Ca, c). Reduced expression of Tie2 on diabetic LT-HSCs when exposed to diabetic osteoblastic niche cells (FIG. 5Cb) was reversed after exposure to nondiabetic osteoblastic niche cells in normal glucose condition (FIG. 5Cd).

### Example 4

Osteoblastic Niche Cell, LT-HSC, ST-HSC and MPP Cell Frequency in Diabetic and Nondiabetic Mice Bone Marrow.

The percentage of osteoblastic niche cells, LT-HSCs, ST-HSCs and MPPs in Lin-bone marrow cells was compared, and the osteoblastic niche cells and LT-HSCs shown to be significantly reduced and ST-HSCs and MPPs were significantly increased in diabetic mice compared with nondiabetic mice (FIG. 6B).

### Example 5

Localization of Osteoblastic Niche Cells and LT-HSC in Diabetic and Nondiabetic Mice Bone Marrow.

In vivo localization of osteoblastic niche cells and LT-HSC in the bone marrow of diabetic and nondiabetic mice was examined by immunofluorescence staining (FIG. 6C). OPN+ osteoblastic niche cells were located at endosteum in both diabetic and nondiabetic mice (red color, arrows), and CD150-positive LT-HSCs were located adjacent to osteoblastic niche cells (green color, arrow heads).

### Example 6

Functional Abnormality in Hematopoietic Stem Cells in Diabetes.

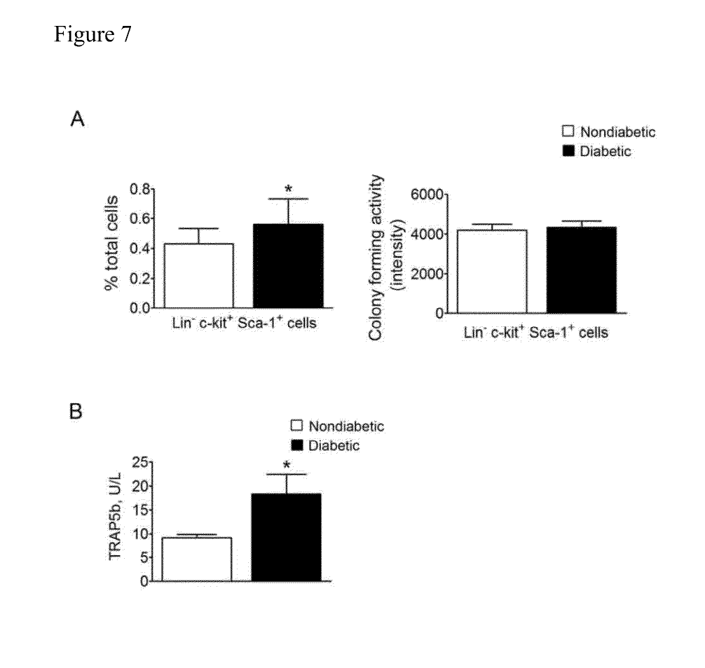

To compare the repopulating activity of hematopoietic stem cells between diabetic and nondiabetic mice, we performed colony forming assays on Lin− c-kit+ Sca-1+ cells. The number of Lin− c-kit+ Sca-1+ cells from diabetic mice was significantly increased compared to nondiabetic mice, however the intensity of colony formation was not changed between diabetic and nondiabetic mice (FIG. 7A). Function of osteoclasts those were derived from hematopoietic progenitors, was also compared between diabetic and non diabetic mice. TRAP5b, indicating the phagocytotic activity of osteoclasts, was significantly increased in diabetic mice compared to nondiabetic mice (FIG. 7B)

### Example 7

Osteoblastic Niche Cell- and LT-HSC-expressing Molecules in Diabetic and Nondiabetic Mice Bone Marrow.

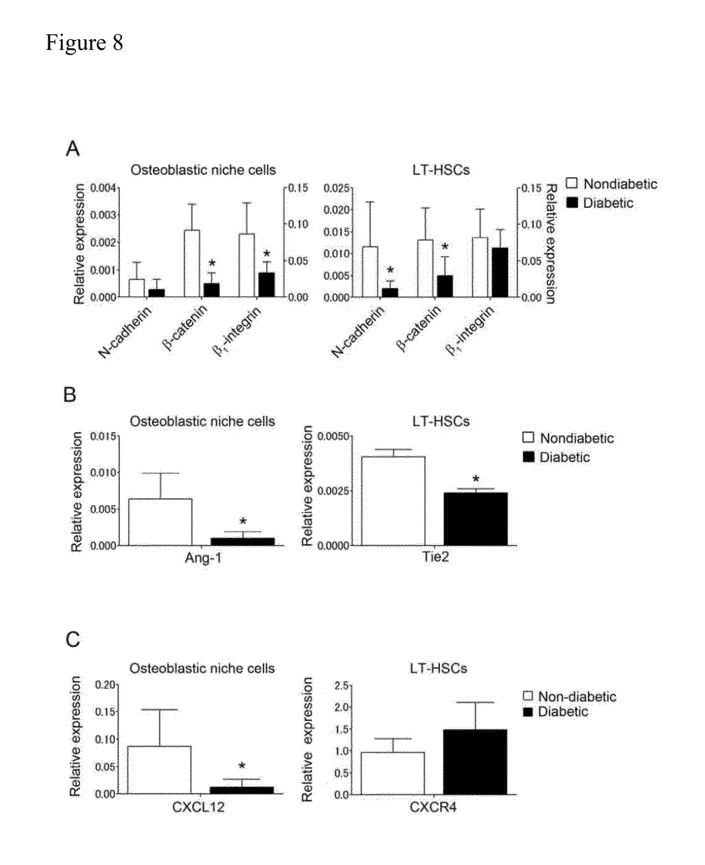

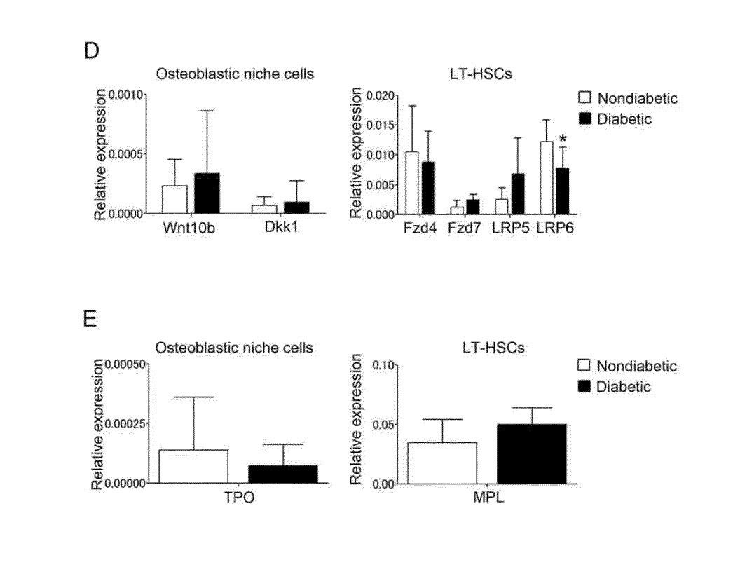

The expression of cell adhesion molecules such as N-cadherin, β-catenin and β1-integrin on isolated osteoblastic niche cells and LT-HSCs from diabetic mice was compared with that from nondiabetic mice. The expression of β-catenin and β1-integrin on isolated osteoblastic niche cells was significantly reduced in diabetic mice, but the expression of N-cadherin was unchanged (FIG. 8A). Diabetic mice showed significantly reduced N-cadherin and β-catenin expression on LT-HSCs compared with nondiabetic mice, while the expression of β1-integrin on LT-HSCs was unchanged (FIG. 8A).

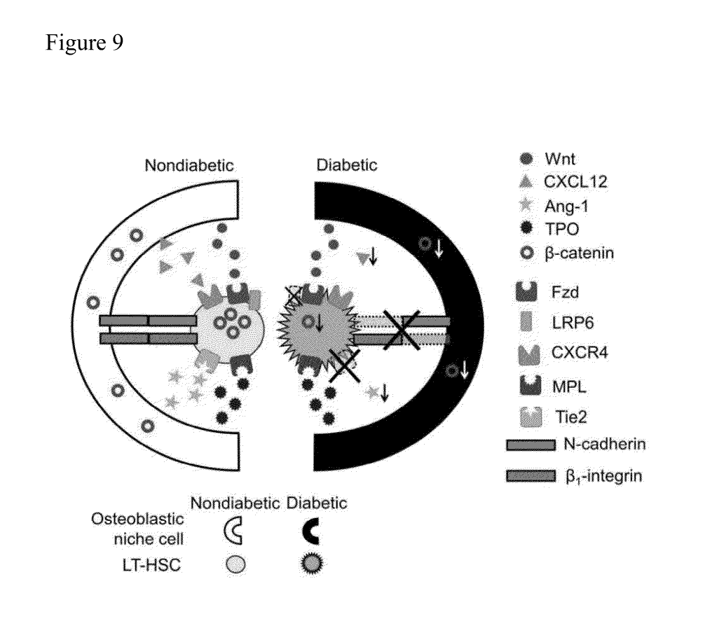

Ang-1 expression on osteoblastic niche cells from diabetic mice was significantly reduced compared with nondiabetic mice, and expression of the Ang-1 ligand Tie2 on LT-HSCs was also significantly reduced in diabetic mice (FIG. 8B). CXCL12 expression on osteoblastic niche cells was significantly reduced in diabetic mice, but expression of its receptor CXCR4 was unchanged on LT-HSCs (FIG. 8C). In terms of Wnt/β-catenin signaling pathways, neither Wnt 10b expression on osteoblastic niche cells nor expression of receptors for Wnt family proteins such as Fzd4, Fzd7 and LRP5 on LT-HSCs was changed; however, LRP6 expression on LT-HSCs was reduced in diabetic mice (FIG. 8D). Dkkl expression was the same on osteoblastic niche cells of both diabetic and nondiabetic mice (FIG. 8D), as was the expression of TPO on osteoblastic niche cells and the expression of its receptor MPL on LT-HSCs (FIG. 8E). A summary of these results is shown in FIG. 9.

### Example 8

In Vitro Co-culture of Osteoblastic Niche Cells and LT-HSCs to Mimic the Bone Marrow Microenvironment in Diabetic and Nondiabetic Mice.

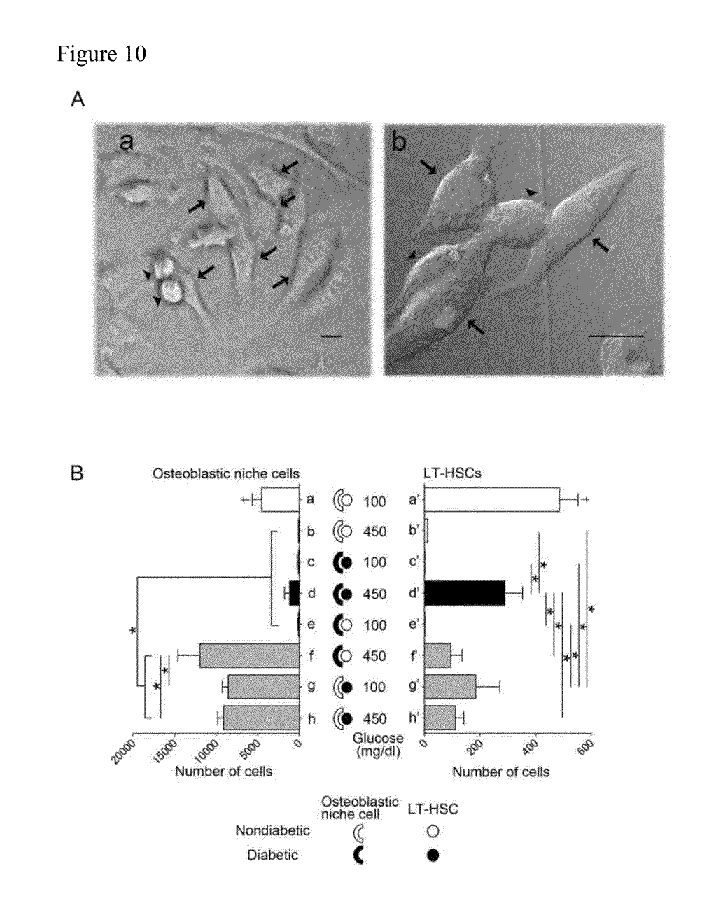

We performed in vitro co-culture experiments of LT-HSCs and osteoblastic niche cells derived from diabetic and nondiabetic mice. After seven days, LT-HSCs were in contact with osteoblastic niche cells at the bottom of the culture plate (FIG. 10Aa, 10Ab). These adherent cells were collected and subsequently isolated into LT-HSCs and osteoblastic niche cells by FACS. As shown in FIG. 10B, osteoblastic niche cells derived from nondiabetic mice kept adherent to the bottom in normal glucose medium but did not keep adherent in high glucose medium (FIG. 10Ba, 10Bb). On the other hand, osteoblastic niche cells derived from diabetic mice kept adherent in high glucose medium but did not keep adherent in normal glucose medium (FIG. 10Bc-f). However osteoblastic niche cells derived from nondiabetic mice co-cultured with LT-HSCs derived from diabetic mice could adhere to the bottom even in high glucose medium (FIG. 10Bh′). The survival frequency of LT-HSCs paralleled the existence of osteoblastic niche cells (FIG. 10Ba′-h′), LT-HSCs survived in a 7-day co-culture system in cases where osteoblastic niche cells kept adherent to the bottoms (FIGS. 10Ba′, d′, f′, g′ and h′).

### Example 9

Changes in the Expression of Molecules on LT-HSCs Co-cultured with Osteoblastic Niche Cells.

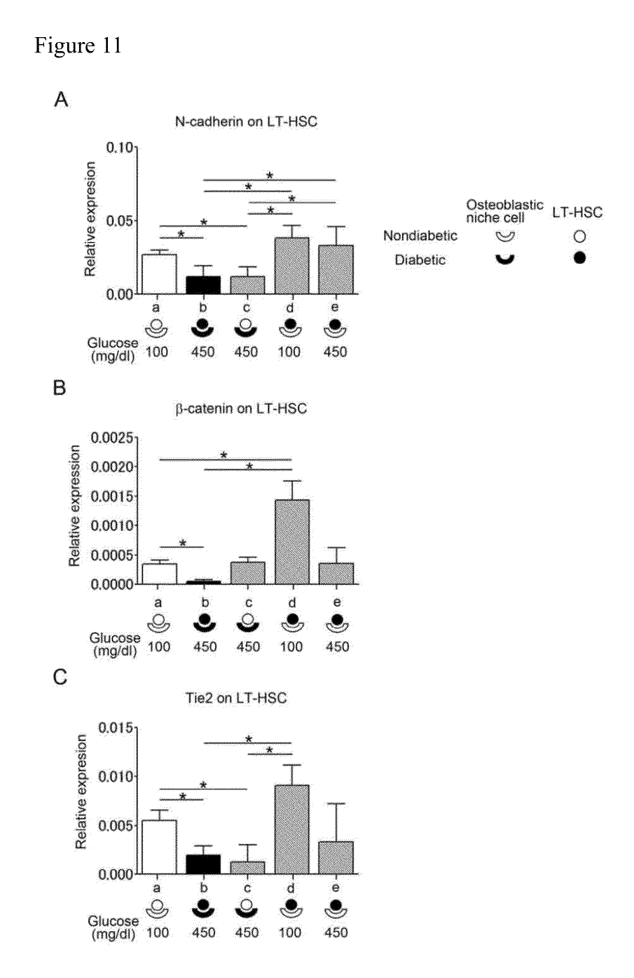

The expression of molecules on LT-HSCs derived from diabetic or nondiabetic mice was examined after co-culture with osteoblastic niche cells derived from diabetic or nondiabetic mice in different glucose conditions. This analysis was only carried out in cases where cells were detected after a 7-day co-culture as shown in FIGS. 10Ba′, 10Bd′, 10Bf′, 10Bg′ and 10Bh′. We analyzed N-cadherin, β-catenin and Tie2 expression on LT-HSCs, as this was shown to be reduced in the in vivo diabetic model. N-cadherin expression on diabetic LT-HSCs exposed to diabetic osteoblastic niche cells was significantly reduced compared with that on nondiabetic LH-HSCs exposed to nondiabetic osteoblastic niche cells (FIG. 11Aa, 11Ab). This was also observed for N-cadherin expression on nondiabetic LT-HSCs exposed to diabetic osteoblastic niche cells under high glucose concentrations compared with those exposed to nondiabetic osteoblastic niche cells (FIG. 11Aa, 11Ac). Reduced expression of N-cadherin on diabetic LT-HSCs exposed to diabetic osteoblastic niche cells (FIG. 11Ab) was, however, reversed after exposure to nondiabetic osteoblastic niche cells in either normal or high glucose conditions (FIG. 11Ad, 11Ae).

Similarly, β-catenin expression on LT-HSCs was examined after in vitro co-culture with osteoblastic niche cells. β-catenin expression on diabetic LT-HSCs exposed to diabetic osteoblastic niche cells was significantly reduced compared with that on nondiabetic LH-HSCs exposed to nondiabetic osteoblastic niche cells (FIG. 11Ba, 11Bb). This was reversed after exposure to nondiabetic osteoblastic niche cells in normal glucose concentrations (FIG. 11Bd).

Tie2 expression on diabetic LT-HSCs exposed to diabetic osteoblastic niche cells was significantly reduced compared with nondiabetic LH-HSCs exposed to nondiabetic osteoblastic niche cells (FIG. 11Ca, 11Cb). This was reversed after exposure to nondiabetic osteoblastic niche cells under normal glucose concentrations (FIG. 11Cd). Tie2 expression on nondiabetic LT-HSCs co-cultured with diabetic osteoblastic niche cells under high glucose concentrations was also significantly reduced compared with those exposed to nondiabetic osteoblastic niche cells (FIG. 11Ca, 11Cc).

### Example 10

Intra Bone Marrow-Bone Marrow Transplantation for In Vivo Replacement of Osteoblastic Niche Cells.

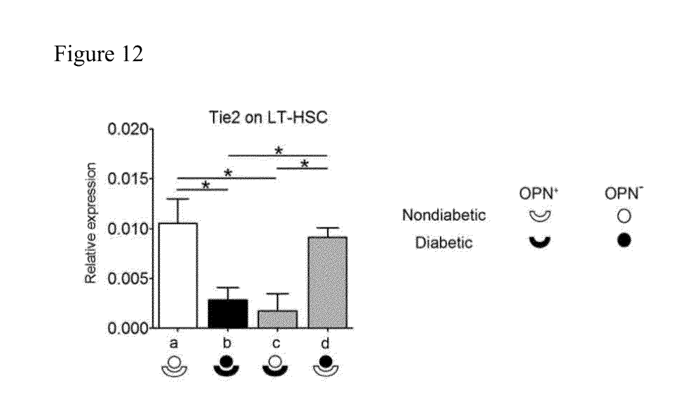

To confirm the findings obtained from in vitro co-culture experiments, we performed intra bone marrow-bone marrow transplantations with OPN+ and OPN− cells of different combinations as follows; nondiabetic OPN+ with nondiabetic OPN− (FIG. 12a), diabetic OPN+ with diabetic OPN− (FIG. 12b), diabetic OPN+ with nondiabetic OPN− (FIG. 12c) and nondiabetic OPN+ with diabetic OPN− cells (FIG. 12d). One week after the bone marrow transplantation, the message level of Tie2 expression on LT-HSCs was examined The results showed that reduced expression of Tie2 on diabetic LT-HSCs exposed to diabetic osteoblastic niche cells (FIG. 12b) was completely revered by exposure to nondiabetic osteoblastic niche cells (FIG. 12d). On the other hand, Tie2 expression on nondiabetic LT-HSCs was significantly reduced by exposure to diabetic osteoblastic niche cells (FIG. 12c).

### REFERENCES

1Fadini G P, Boscaro E, de Kreutzenberg S, Agostini C, Seeger F et al. (2010) Time course and mechanisms of circulating progenitor cell reduction in the natural history of type 2 diabetes. Diabetes Care 33: 1097-1102.
2 Tepper O M, Can J, Allen R J Jr, Chang C C, Lin C D et al. (2010) Decreased circulating progenitor cell number and failed mechanisms of stromal cell-derived factor-lalpha mediated bone marrow mobilization impair diabetic tissue repair. Diabetes 59: 1974-1983.
3 Orlandi A, Chavakis E, Seeger F, Tjwa M, Zeiher AM et al. (2010) Long-term diabetes impairs repopulation of hematopoietic progenitor cells and dysregulates the cytokine expression in the bone marrow microenvironment in mice. Basic Res Cardiol 105: 703-712.
4 Arai F, Suda T. (2007) Maintenance of quiescent hematopoietic stem cells in the osteoblastic niche. Ann N.Y. Acad Sci 106:41-53.
5 Omatsu Y, Sugiyama T, Kohara H, Kondoh G, Fujii N et al. (2010)The essential functions of adipo-osteogenic progenitors as the hematopoietic stem and progenitor cell niche. Immunity 33: 387-399.
6 Méndez-Ferrer S, Michurina T V, Ferraro F, Mazloom A R, Macarthur B D et al. (2010) Mesenchymal and haematopoietic stem cells form a unique bone marrow niche. Nature 466: 829-834.
7 Ehninger A, Trumpp A. (2011) The bone marrow stem cell niche grows up: mesenchymal stem cells and macrophages move in. J Exp Med 208: 421-428.
8 Ferraro F, Lymperi S, Mendez-Ferrer S, Saez B, Spencer J A, Yeap BY et al. (2011) Diabetes impairs hematopoietic stem cell mobilization by altering niche function. Sci Transl Med 3: 101-104.
9 Mercier F E, Ragu C, Scadden D T (2012) The bone marrow at the crossroads of blood and immunity. Nature Review Immunology 12: 49-60.
10 Wang D L, Wagers A J (2011) Dynamic niches in the origination and differentiation of haematopoietic stem cells. Nature Review Molecular Cell Biol 12: 643-655.
11 Chan L, Terashima T, Fujimiya M, Kojima H (2006) Chronic diabetic complications: the body's adaptive response to hyperglycemia gone awry? Trans Am Clin Climatol Assoc 117: 341-351.
12 Fujimiya M, Kojima H, Ichinose M, Arai R, Kimura H et al. (2007) Fusion of proinsulin-producing bone marrow-derived cells with hepatocytes in diabetes. Proc Natl Acad Sci U S A 104: 4030-4035.
13 Terashima T, Kojima H, Fujimiya M, Matsumura K, Oi J et al. (2005) The fusion of bone-marrow-derived proinsulin-expressing cells with nerve cells underlies diabetic neuropathy. Proc Natl Acad Sci USA 102: 12525-12530.
14 Yamashita T, Fujimiya M, Nagaishi K, Ataka K, Tanaka M et al. (2011) Fusion of bone marrow-derived cells with renal tubules contributes to renal dysfunction in diabetic nephropathy. FASEB J (in press).
15 Mayack S R, Shadrach J L, Kim F S, Wagers A J (2010) Systemic signals regulate ageing and rejuvenation of blood stem cell niches. Nature 463: 495-500.
16 Kawamoto T. (2003) Use of a new adhesive film for the preparation of multi-purpose fresh-frozen sections from hard tissues, whole-animals, insects and plants. Arch Histol Cytol. 66: 123-43.
17 Weksberg D C, Chambers S M, Boles N C, Goodell M A. (2008) CD150- side population cells represent a functionally distinct population of long-term hematopoietic stem cells. Blood 111: 2444-51
18 Kushida T, Inaba M, Hisha H, Ichioka N, Esumi T, Ogawa R, Iida H, Ikehara S. (2001) Intra-bone marrow injection of allogeneic bone marrow cells: a powerful new strategy for treatment of intractable autoimmune diseases in MRL/lpr mice. Blood. 97: 3292-9.
19 Li L, Bhatia R Stem cell quiescence. (2011) Clin Cancer Res 17: 4936-4941.
20 Bar-Shavit Z. (2007) The osteoclast: a multinucleated, hematopoietic-origin, bone-resorbing osteoimmune cell. J Cell Biochem. 102: 1130-9.
21 Motyl K, McCabe L R. (2009) Streptozotocin, type I diabetes severity and bone. Biol Proced Online 11: 296-315.
22 Hosokawa K, Arai F, Yoshihara H, Iwasaki H, Nakamura Y et al. (2010) Knockdown of N-cadherin suppresses the long-term engraftment of hematopoietic stem cells. Blood 116: 554-563.
23 Haug J S, He X C, Grindley J C, Wunderlich J P, Gaudenz K et al. N-cadherin expression level distinguished reserved versus primed states of hematopoietic stem cells. Cell Stem Cells (2008) 2: 367-379.
24 Gottschling S, Saffrich R, Seckinger A, Krause U, Horsch K et al. (2007) Human mesenchymal stromal cells regulate initial self-renewing divisions of hematopoietic progenitor cells by a betal-integrin-dependent mechanism. Stem Cells 25:798-806.
25 Baron R, Rawadi G (2007) Targeting the Wnt/beta-catenin pathway to regulate bone formation in the adult skeleton. Endocrinology 148: 2635-2643.
26 Kirstetter P, Anderson K, Porse B T, Jacobsen S E, Nerlov C et al. (2006) Activation of the canonical Wnt pathway leads to loss of hematopoietic stem cell repopulation and multilineage differentiation block. Nature Immunology 7: 1048-1056.
27 Fleming H E, Janzen V, Lo Celso C, Guo J, Leahy K M et al. (2008) Wnt signaling in the niche enforces hematopoietic stem cell quiescence and is necessary to preserve self-renewal in vivo. Cell Stem Cell 2: 274-283.
28 Suda T, Arai F (2008) Wnt signaling in the niche. Cell 132: 729-730.
29 Tzeng Y S, Li H, Kang Y L, Chen W C, Cheng W C et al. (2011) Loss of Cxcl12/Sdf-1 in adult mice decreases the quiescent state of hematopoietic stem/progenitor cells and alters the pattern of hematopoietic regeneration after myelosuppression. Blood 117: 429-439.
30 Zou Y R, Kottmann A H, Kuroda M, Taniuchi I, Littman D R et al. (1998) Function of the chemokine receptor CXCR4 in haematopoiesis and in cerebellar development. Nature 393: 595-599.
31 Leisten I, Kramann R, Ventura Ferreira M S, Bovi M, Neuss S et al. (2012) 3D co-culture of hematopoietic stem and progenitor cells and mesenchymal stem cells in collagen scaffolds as a model of the hematopoietic niche. Biomaterials 3: 1736-1747.
32 Sharma M B, Limaye L S, Kale V P. (2011) Mimicking the functional hematopoietic stem cell niche in vitro: recapitulation of marrow physiology by hydrogel-based three-dimensional cultures of mesenchymal stromal cells. Hematologica (in press).
33 Jing D, Wobus M, Poitz D M, Bornhauser M, Ehninger G et al. (2011) Oxygen tension plays a critical role in the hematopoieticmicroenvironment in vitro. Hematologica (in press).

What is claimed is:

1. A method of establishing in vitro co-culture of LT-HSCs and osteoblastic niche cells, comprising the steps of:
isolating LT-HSCs;
isolating osteoblastic niche cells;
mixing said isolated LT-HSCs and said isolated osteoblastic niche cells while transferring them to a tissue culture dish;
culturing the cell mixture until LT-HSCs and osteoblastic niche cells attach together on the culture dish to form 2-dimensional co-culture system, which is maintained more than 3 days while preserving both viability of the osteoblastic niche cells and the immature phenotype of the LT-HSCs.

2. The method of claim 1, wherein the phenotype of said osteoblastic niche cells is; Wnt⁺, CXCL-12⁺, TPO⁺, Ang-1⁺, N-cadherin⁺, β-catenin⁺, β1-integrin⁺, osteopontin⁺, CD45⁻, and Lin⁻, and wherein LT-HSCs and osteoblastic niche cells are isolated from bone marrow.

3. A method of screening people suffering from a disorder caused by dysfunction of osteoblastic niche cells, comprising either:
(a) isolating LT-HSCs from the subject for which presence of disorder is to be determined, then
(b) detecting in said LT-HSCs differential expression of at least one gene selected from the group consisting of: N-cadherin, LRP6, β-catenin, and Tie2, wherein downregulation of expression of said at least one gene indicates presence of said disorder, or
(c) isolating osteoblastic niche cells from the subject for which presence of disorder is to be determined, then
(d) detecting in said osteoblastic niche cells differential expression of at least one gene selected from the group consisting of: CXCL-12, Ang-1, β-catenin and β1-integrin;
wherein downregulation of expression of said at least one gene indicates presence of said disorder.

4. A method of screening a candidate substance for treatment of a disorder caused by dysfunction of osteoblastic niche cells, comprising:
(a) providing 2-dimensional co-culture system of LT-HSCs and osteoblastic niche cells according to the method of claim 1, then
(b) exposing said co-culture system to a test substance; and then
(c) detecting either in LT-HSCs of the culture system differential expression of at least one gene selected from the group consisting of N-cadherin, LRP6, β-catenin, and Tie2, or in osteoblastic niche cells of the culture system differential expression of at least one gene selected from the group consisting of CXCL-12, Ang-1, β-catenin and β1-integrin, and wherein upregulation of expression of said at least one gene indicates that the reagent is a candidate reagent for said disorder.

5. The method of claim 3, wherein said disorder is selected from groups of disorders including: autoimmune diseases including nephrosis, glomerulonephritis, type 1 and type 2 diabetes mellitus and their complications, vitiligo, Crohn's disease, ulcerative colitis, Behcet's syndrome, and psoriasis; collagen diseases including chronic rheumatism, scleroderma, systemic lupus erythematosus, and dermatomyositis; blood disorders including leukemia, aplastic anemia, and myelodysplastic syndrome; degenerative diseases including Alzheimer's disease, multiple sclerosis, amyotrophic lateral sclerosis, and osteoporosis; chronic inflammations including arteriosclerosis, emphysema, chronic obstructive pulmonary disease, hepatic cirrhosis, and atopic dermatitis; and malignant tumors.

6. The method of claim 4, wherein said disorder is selected from groups of disorders including: autoimmune diseases including nephrosis, glomerulonephritis, type 1 and type 2 diabetes mellitus and their complications, vitiligo, Crohn's disease, ulcerative colitis, Behcet's syndrome, and psoriasis; collagen diseases including chronic rheumatism, scleroderma, systemic lupus erythematosus, and dermatomyositis; blood disorders including leukemia, aplastic anemia, and myelodysplastic syndrome; degenerative diseases including Alzheimer's disease, multiple sclerosis, amyotrophic lateral sclerosis, and osteoporosis; chronic inflammations including arteriosclerosis, emphysema, chronic obstructive pulmonary disease, hepatic cirrhosis, and atopic dermatitis; and malignant tumors.

7. Method of treating a patient suffering from a disorder caused by dysfunction of osteoblastic niche cells, comprising:
(a) providing osteoblastic niche cells of which expression of at least one gene selected from the group consisting of: CXCL-12, Ang-1, β-catenin and β1-integrin, is within normal levels, and then
(b) administrating an effective amount of said osteoblastic niche cells.

8. The method of claim 7, wherein said normal levels mean between half and twice of the average mRNA expression level of the corresponding gene for healthy people.

9. The method of claim 7, wherein said osteoblastic niche cells are allogeneic to the patient.

10. The method of claim 9, wherein said osteoblastic niche cells are checked for CXCL-12, Ang-1, β-catenin or β1-integrin to be within normal levels

11. The method of claim 7, wherein said osteoblastic niche cells are collected from the patient.

12. The method of claim 11, wherein said osteoblastic niche cells collected from the patient are co-cultured according to claim 1 with LT-HSCs which are collected from a healthy donor.
